(19) United States
(12) Patent Application Publication
(10) Pub. No.: US 2003/0003073 A1
(43) Pub. Date: Jan. 2, 2003

(54) Hair conditioning composition comprising a fatty alcohol mixture, and cosmetic process for treating the hair

(76) Inventor: Rainer Muller, Leopoldshafen (DE)

Correspondence Address: NIXON & VANDERHYE P.C., 8th Floor, 1100 North Glebe Road, Arlington, VA, 22201-4714, US

(21) Appl. No.: 10/143,764

(22) Filed: May 14, 2002

(30) Foreign Application Priority Data
May 15, 2001 (FR) 0106383

(51) Int. Cl. A61K 7/075

(52) U.S. Cl. 424/70.31

(57) ABSTRACT

The present invention relates to a hair conditioning composition comprising, in a cosmetically acceptable aqueous medium, at least one cationic surfactant, a fatty alcohol mixture that essentially comprises C₂₀₋₂₄ fatty alcohols, and optionally detergent surfactants in an amount of less than or equal to 4% by weight relative to the total weight of the composition. This composition may be used in particular as an after-shampoo conditioner on fine hair.

The present invention relates to a hair conditioning composition comprising a particular fatty alcohol mixture, and to a cosmetic process for treating the hair.

After-shampoo conditioners are conditioning compositions that are well known in the art. They are generally fatty alcohol emulsions.

The fatty alcohols thus used are well known to those skilled in the art as being, especially, conditioners and/or thickeners.

Specifically, saturated long-chain primary alcohols containing on average from 25 to 45 carbon atoms are described in documents U.S. Pat. Nos. 5,213,716 and 5,348,736 from Colgate.

Documents WO 99/62467 and WO 99/62492 from Procter & Gamble also describe hair conditioning compositions comprising, inter alia, a mixture of cetyl alcohol, stearyl alcohol and behenyl alcohol, the behenyl alcohol constituting up to 15% of the alcohol mixture.

After applying the after-shampoo conditioners of the prior art, in particular on fine hair, the Applicant found that the hair was charged and lacked lightness, especially after repeated use.

The Applicant has thus sought to obtain a less substantial deposit of fatty alcohols and of conditioning agents on fine hair, while at the same time conserving good conditioning properties such as suppleness and a smooth nature for wet hair, and manageability for dry hair.

The Applicant has discovered, surprisingly, that by limiting the length of the carbon chain of the fatty alcohols in the after-shampoo conditioners, to a number of carbon atoms of between 20 and 24, it is possible to reduce the fatty alcohol deposit on the hair and also the deposit of conditioning agents that co-precipitate with the fatty alcohol, such as, for example, silicones and cationic surfactants. These compositions thus make it possible to overcome the drawbacks described above and to obtain wet hair that is more supple and smoother, and also dried hair that is more manageable and more individualized.

One subject of the present invention is thus a hair conditioning composition comprising, in a cosmetically acceptable aqueous medium, at least one cationic surfactant and a fatty alcohol mixture as described below.

Another subject of the invention consists of a cosmetic process for treating the hair using the abovementioned composition.

A subject of the invention is also the use of the said composition as an after-shampoo conditioner.

Other subjects, characteristics, aspects and advantages of the invention will emerge even more clearly on reading the description and the various examples that follow.

According to the invention, the hair conditioning composition comprises, in a cosmetically acceptable aqueous medium, at least one cationic surfactant, a fatty alcohol mixture that essentially comprises C₂₀₋₂₄ fatty alcohols, and optionally detergent surfactants in an amount of less than or equal to 4% by weight relative to the total weight of the composition.

The term “essentially” means a fatty alcohol mixture comprising at least 60% by weight, preferably at least 80% by weight and more particularly at least 90% by weight, of C₂₀₋₂₄ fatty alcohols relative to the total weight of the mixture.

The C₂₀₋₂₄ fatty alcohols that may be used in the alcohol mixture of the present invention comprise a linear or branched, saturated or unsaturated hydrocarbon-based chain. They are preferably primary fatty alcohols with a saturated linear chain. Examples that may be mentioned especially include behenyl alcohol, arachidyl alcohol and lignoceryl alcohol, and mixtures thereof.

The composition according to the invention can also contain an amount of less than or equal to 40% by weight, preferably less than or equal to 20% by weight and more particularly less than or equal to 10% by weight, relative to the total weight of the mixture, of at least one C₁₄₋₁₉ alcohol such as, for example, cetyl alcohol or stearyl alcohol.

In addition, the composition according to the invention can contain an amount of less than or equal to 40% by weight, preferably less than or equal to 20% by weight and more particularly less than or equal to 10% by weight, relative to the total weight of the mixture, of at least one C₂₅₋₄₀ alcohol such as, for example, ceryl alcohol or montanyl alcohol.

As a fatty alcohol mixture that is particularly preferred in the composition according to the invention, it is possible to use, for example, the fatty alcohol mixture consisting of 76% by weight behenyl alcohol, 17% by weight arachidyl alcohol, 1.5% by weight lignoceryl alcohol, 5% by weight stearyl alcohol and 0.5% by weight cetyl alcohol. This mixture is sold under the name Nafol® 1822 C by the company Condea. Other examples that may also be mentioned include the mixture sold under the name Nafol® 2298 by the company Condea, which comprises 98% by weight behenyl alcohol; the mixture sold under the name Nafol® 20-22 by the company Condea, which comprises 30% by weight behenyl alcohol, 58% by weight arachidyl alcohol and 6% by weight lignoceryl alcohol; or the mixture sold under the name Nafol® 20+ by the company Condea, which comprises 50% by weight arachidyl alcohol, 29% by weight behenyl alcohol, 14% by weight lignoceryl alcohol and 6% by weight stearyl alcohol.

The amount of the fatty alcohol mixture in the composition according to the invention is especially between 0.1% and 20% by weight and preferably between 0.5% and 10% by weight relative to the total weight of the composition.

The composition according to the invention comprises one or more cationic surfactants that are well known per se, such as salts of optionally polyoxyalkylenated primary, secondary or tertiary fatty amines and quaternary ammonium salts, and mixtures thereof.

Examples of quaternary ammonium salts that may especially be mentioned include:

those of general formula (I) below:

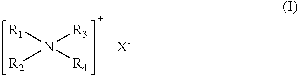

in which the symbols R₁ to R₄, which may be identical or different, represent a linear or branched aliphatic radical containing from 1 to 30 carbon atoms, or an aromatic radical such as aryl or alkylaryl. The aliphatic radicals can comprise hetero atoms such as, in particular, oxygen, nitrogen, sulphur or halogens. The aliphatic radicals are chosen, for example, from alkyl, alkoxy, polyoxy(C₂-C₆)alkylene, alkylamide, (C₁₂-C₂₂)alkylamido(C₂-C₆)alkyl, (C₁₂-C₂₂) alkylacetate and hydroxyalkyl radicals, comprising from about 1 to 30 carbon atoms; X⁻ is an anion chosen from the group of halides, phosphates, acetates, lactates, (C₂-C₆)alkyl sulphates and alkyl or alkylaryl sulphonates;

quaternary ammonium salts of imidazoline, such as, for example, those of formula (II) below:

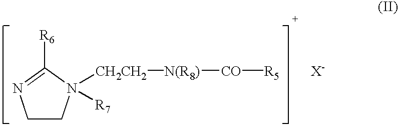

in which R₅ represents an alkenyl or alkyl radical containing from 8 to 30 carbon atoms, for example tallow fatty acid derivatives, R₆ represents a hydrogen atom, a C₁-C₄ alkyl radical or an alkenyl or alkyl radical containing from 8 to 30 carbon atoms, R₇ represents a C₁-C₄ alkyl radical, R₈ represents a hydrogen atom or a C₁-C₄ alkyl radical, X⁻ is an anion chosen from the group of halides, phosphates, acetates, lactates, alkyl sulphates and alkyl or alkylaryl sulphonates. R₅ and R₆ preferably denote a mixture of alkenyl or alkyl radicals containing from 12 to 21 carbon atoms, for example tallow fatty acid derivatives, R₇ denotes a methyl radical and R₈ denotes a hydrogen atom. Such a product is sold, for example, under the name Rewoquat® W 75 by the company Rewo;

diquaternary ammonium salts of formula (III):

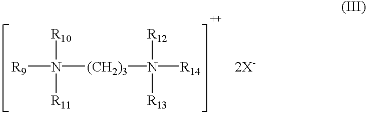

in which R₉ denotes an aliphatic radical containing from about 16 to 30 carbon atoms, R₁₀, R₁₁, R₁₂, R₁₃ and R₁₄, which may be identical or different, are chosen from hydrogen or an alkyl radical containing from 1 to 4 carbon atoms, and X⁻ is an anion chosen from the group of halides, acetates, phosphates, nitrates and methyl sulphates. Such diquaternary ammonium salts in particular comprise propane tallow diammonium dichloride;

quaternary ammonium salts containing at least one ester function, such as those of formula (IV) below:

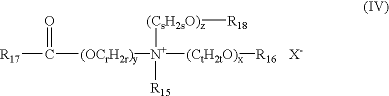

in which:

R₁₅ is chosen from C₁-C₆ alkyl radicals and C₁-C₆ hydroxyalkyl or dihydroxyalkyl radicals;

R₁₆ is chosen from:

a radical

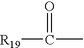

linear or branched, saturated or unsaturated C₁-C₂₂ hydrocarbon-based radicals R₂₀,

a hydrogen atom,

R₁₇ is chosen from:

a radical

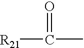

linear or branched, saturated or unsaturated C₁-C₆ hydrocarbon-based radicals R₂₂,

a hydrogen atom,

R₁₇, R₁₉ and R₂₁, which may be identical or different, are chosen from linear or branched, saturated or unsaturated C₇-C₂₁ hydrocarbon-based radicals;

r, s and t, which may be identical or different, are integers ranging from 2 to 6;

y is an integer ranging from 1 to 10;

x and z, which may be identical or different, are integers ranging from 0 to 10;

X⁻ is a simple or complex, organic or inorganic anion;

with the proviso that the sum x+y+z is from 1 to 15, that when x is 0, then R₁₆ denotes R₂₀ and that when z is 0, then R₁₈ denotes R₂₂.

The R₁₅ alkyl radicals may be linear or branched and more particularly linear.

R₁₅ preferably denotes a methyl, ethyl, hydroxyethyl or dihydroxypropyl radical and more particularly a methyl or ethyl radical.

The sum x+y+z is advantageously from 1 to 10.

When R₁₆ is a hydrocarbon-based radical R₂₀, it may be long and contain from 12 to 22 carbon atoms, or short and contain from 1 to 3 carbon atoms.

When R₁₈ is a hydrocarbon-based radical R₂₂, it preferably contains 1 to 3 carbon atoms.

R₁₇, R₁₉ and R₂₁, which may be identical or different, are advantageously chosen from linear or branched, saturated or unsaturated C₁₁-C₂₁ hydrocarbon-based radicals, and more particularly from linear or branched, saturated or unsaturated, C₁₁-C₂₁ alkyl and alkenyl radicals.

x and z, which may be identical or different, are preferably 0 or 1.

y is advantageously equal to 1.

r, s and t, which may be identical or different, are preferably 2 or 3 and even more particularly are equal to 2.

The anion is preferably a halide (chloride, bromide or iodide) or an alkyl sulphate, more particularly methyl sulphate. However, methanesulphonate, phosphate, nitrate, tosylate, an anion derived from an organic acid, such as acetate or lactate, or any other anion that is compatible with the ammonium containing an ester function, may be used.

The anion X⁻ is even more particularly chloride or methyl sulphate.

The ammonium salts more particularly used in the composition according to the invention are those of formula (IV) in which:

R₁₅ denotes a methyl or ethyl radical,

x and y are equal to 1;

z is equal to 0 or 1;

r, s and t are equal to 2;

R₁₆ is chosen from:

a radical

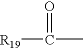

methyl, ethyl or C₁₄-C₂₂ hydrocarbon-based radicals;

a hydrogen atom;

R₁₈ is chosen from:

a radical

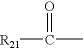

a hydrogen atom;

R₁₇, R₁₉ and R₂₁, which may be identical or different, are chosen from linear or branched, saturated or unsaturated C₁₃-C₁₇ hydrocarbon-based radicals and preferably from linear or branched, saturated or unsaturated C₁₃-C₁₇ alkyl and alkenyl radicals.

The hydrocarbon-based radicals are advantageously linear.

Examples that may be mentioned include the compounds of formula (IV) such as the diacyloxyethyldimethylammonium, diacyloxyethylhydroxyethylmethylammonium, monoacyloxyethyldihydroxyethylmethylammonium, triacyloxyethylmethylammonium and monoacyloxyethylhydroxyethyldimethylammonium salts (chloride or methyl sulphate in particular), and mixtures thereof. The acyl radicals preferably contain 14 to 18 carbon atoms and are obtained more particularly from a plant oil such as palm oil or sunflower oil. When the compound contains several acyl radicals, these radicals may be identical or different.

These products are obtained, for example, by direct esterification of triethanolamine, triisopropanolamine, an alkyldiethanolamine or an alkyldiisopropanolamine, which are optionally oxyalkylenated, with fatty acids or with fatty acid mixtures of plant or animal origin, or by transesterification of the methyl esters thereof. This esterification is followed by a quaternization using an alkylating agent such as an alkyl halide (preferably a methyl or ethyl halide), a dialkyl sulphate (preferably dimethyl or diethyl sulphate), methyl methanesulphonate, methyl para-toluenesulphonate, glycol chlorohydrin or glycerol chlorohydrin.

Such compounds are sold, for example, under the names Dehyquart® by the company Cognis, Stepanquat® by the company Stepan, Noxamium® by the company CECA or Rewoquat® WE 18 by the company Rewo-Goldschmidt.

The composition according to the invention may preferably contain a mixture of quaternary ammonium salts of mono-, di- and triesters with a weight majority of diester salts.

Examples of mixtures of ammonium salts that may be used include the mixture containing 15% to 30% by weight of acyloxyethyldihydroxyethylmethylammonium methyl sulphate, 45% to 60% of diacyloxyethylhydroxyethylmethylammonium methyl sulphate and 15% to 30% of triacyloxyethylmethylammonium methyl sulphate, the acyl radicals containing from 14 to 18 carbon atoms and being derived from palm oil that is optionally partially hydrogenated.

It is also possible to use the ammonium salts containing at least one ester function that are described in patents U.S. Pat. Nos. 4,874,554 and 4,137,180.

Among the quaternary ammonium salts mentioned above, the ones preferably used are those corresponding to formula (I). The ones that may especially be mentioned are, on the one hand, tetraalkylammonium chlorides such as, for example, dialkyldimethylammonium chlorides or alkyltrimethylammonium chlorides, in which the alkyl radical contains from about 12 to 22 carbon atoms, in particular behenyltrimethylammonium chloride, distearyldimethylammonium chloride, cetyltrimethylammonium chloride, or benzyldimethylstearylammonium chloride, or, on the other hand, palmitylamidopropyltrimethylammonium chloride or stearamidopropyldimethyl (myristyl acetate) ammonium chloride sold under the name Ceraphyl® 70 by the company Van Dyk.

The cationic surfactants that are particularly preferred in the composition of the invention are chosen from quaternary ammonium salts, and in particular from behenyltrimethylammonium chloride, cetyltrimethylammonium chloride and palmitylamidopropyltrimethylammonium chloride.

The composition according to the invention preferably contains the cationic surfactants in an amount of from 0.05% to 10% by weight and preferably from 0.1% to 5% by weight relative to the total weight of the composition.

The composition according to the invention may optionally contain an amount of less than or equal to 4% by weight of detergent surfactants chosen from anionic, nonionic and amphoteric surfactants, and mixtures thereof.

The detergent surfactants that may be used in the present invention are chosen from the standard anionic, nonionic and amphoteric surfactants that are well known in the art, and mixtures thereof.

The composition according to the invention may also comprise at least one conditioner chosen from silicones, cationic polymers, esters, plant oils, mineral oils and synthetic oils such as poly(α-olefins), and mixtures thereof.

The silicones that may be used in accordance with the invention may be soluble or insoluble in the composition, and they may be in particular polyorganosiloxanes that are insoluble in the composition of the invention. They may be in the form of oils, waxes, resins or gums. They may be used pure or as an emulsion, a dispersion or a microemulsion.

The organopolysiloxanes are defined in greater detail in Walter Noll's “Chemistry and Technology of Silicones” (1968) Academic Press. They can be volatile or non-volatile.

When they are volatile, the silicones are more particularly chosen from those having a boiling point of between 60° C. and 260° C., and even more particularly from:

(i) cyclic silicones containing from 3 to 7 and preferably from 4 to 5 silicon atoms. These are, for example, octamethylcyclotetrasiloxane sold in particular under the name “Volatile Silicone 7207” by Union Carbide or “Silbione 70045 V 2” by Rhodia, decamethylcyclopentasiloxane sold under the name “Volatile Silicone 7158” by Union Carbide, and “Silbione 70045 V 5”, by Rhodia, and mixtures thereof.

Mention may also be made of cyclocopolymers of the dimethylsiloxane/methylalkylsiloxane type, such as “Volatile Silicone FZ 3109” sold by the company Union Carbide, having the chemical structure:

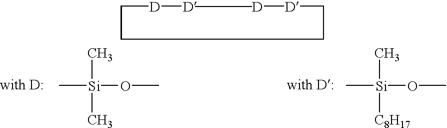

Mention may also be made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;

(ii) linear volatile silicones containing 2 to 9 silicon atoms and having a viscosity of less than or equal to 5×10⁻⁶ m²/s at 25° C. An example is decamethyltetrasiloxane sold in particular under the name “SH 200”, by the company Toray Silicone. Silicones belonging to this category are also described in the article published in Cosmetics and Toiletries, Vol. 91, January 76, pp. 27-32, Todd & Byers “Volatile Silicone Fluids for Cosmetics”.

Among the non-volatile silicones that may be mentioned especially are polyalkylsiloxanes, polyarylsiloxanes, polyalkylarylsiloxanes, silicone gums and resins, and polyorganosiloxanes modified with organofunctional groups, and also mixtures thereof.

The organomodified silicones that may be used in accordance with the invention are silicones as defined above and containing in their structure one or more organofunctional groups attached via a hydrocarbon-based radical.

Among the organomodified silicones that may be mentioned are polyorganosiloxanes containing:

polyethyleneoxy and/or polypropyleneoxy groups optionally containing C₆-C₂₄ alkyl groups, such as the products known as dimethicone copolyol sold by the company Dow Corning under the name DC 1248 or the oils Silwet® L 722, L 7500, L 77 and L 711 from the company Union Carbide and the (C₁₂)alkylmethicone copolyol sold by the company Dow Corning under the name Q2 5200;

substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups are, in particular, C₁-C₄ aminoalkyl groups;

thiol groups such as the products sold under the names “GP 72 A ” and “GP 71” from Genesee;

alkoxylated groups such as the product sold under the name “Silicone Copolymer F-755” by SWS Silicones and Abil Wax® 2428, 2434 and 2440 by the company Goldschmidt;

hydroxylated groups such as the polyorganosiloxanes containing a hydroxyalkyl function, described in French patent application FR-A-85/16334;

acyloxyalkyl groups such as, for example, the polyorganosiloxanes described in patent U.S. Pat. No. 4,957,732;

anionic groups of the carboxylic acid type, such as, for example, in the products described in patent EP 186 507 from the company Chisso Corporation, or of alkylcarboxylic type, such as those present in the product X-22-3701E from the company Shin-Etsu; 2-hydroxyalkyl sulphonate; 2-hydroxyalkyl thiosulphate such as the products sold by the company Goldschmidt under the names “Abil® S201” and “Abil® S255”.

hydroxyacylamino groups, such as the polyorganosiloxanes described in patent application EP 342 834. Mention may be made, for example, of the product Q2-8413 from the company Dow Corning.

Examples of silicones that are preferably used include polydimethylsiloxanes, polyalkylarylsiloxanes and polydimethylsiloxanes containing amino or alkoxy groups.

The composition according to the invention may also comprise one or more cationic polymers. The expression “cationic polymer” means any polymer containing cationic groups and/or groups that may be ionized into cationic groups.

The cationic polymers that may be used in accordance with the present invention may be chosen from any of those already known per se as improving the cosmetic properties of hair treated with detergent compositions, namely, in particular, those described in patent application EP-A-0 337 354 and in French patent applications FR-A-2 270 846, 2 383 660, 2 598 611, 2 470 596 and 2 519 863.

The preferred cationic polymers are chosen from those that contain units including primary, secondary, tertiary and/or quaternary amine groups, that may either form part of the main polymer chain, or be borne by a side substituent directly linked thereto.

The cationic polymers used generally have a number-average molecular mass of between 500 and 5×10⁶ approximately and preferably between 10³ and 3×10⁶ approximately.

Among the cationic polymers that may be mentioned more particularly are polymers of the polyamine, polyamino amide and polyquaternary ammonium type. These are known products.

The polymers of the polyamine, polyamino amide and polyquaternary ammonium type that may be used in the composition of the present invention are those described in French patents 2 505 348 and 2 542 997. Among these polymers, mention may be made of:

(1) homopolymers or copolymers derived from acrylic acid or methacrylic acid esters or amides;

(2) the cellulose ether derivatives containing quaternary ammonium groups, described in French patent 1 492 597;

(3) cationic cellulose derivatives such as cellulose copolymers or cellulose derivatives grafted with a water-soluble quaternary ammonium monomer, and described in particular in U.S. Pat. No. 4,131,576, such as hydroxyalkylcelluloses, for instance hydroxymethyl-, hydroxyethyl- or hydroxypropylcelluloses grafted, in particular, with a methacryloylethyltrimethylammonium, methacrylamidopropyltrimethylammonium or dimethyldiallylammonium salt;

(4) the cationic polysaccharides described more particularly in U.S. Pat. Nos. 3,589,578 and 4,031,307, such as guar gums containing cationic trialkylammonium groups;

(5) polymers consisting of piperazinyl units and of divalent alkylene or hydroxyalkylene groups containing straight or branched chains, optionally interrupted by oxygen, sulphur or nitrogen atoms or by aromatic or heterocyclic rings, as well as the oxidation and/or quaternization products of these polymers. Such polymers are described, in particular, in French patents 2 162 025 and 2 280 361;

(6) water-soluble polyamino amides such as those described, in particular, in French patents 2 252 840 and 2 368 508;

(7) polyamino amide derivatives, for example, adipic acid/dialkylaminohydroxyalkyldialkylenetriamine polymers in which the alkyl group contains from 1 to 4 carbon atoms and preferably denotes a methyl, ethyl or propyl group, and the alkylene group contains from 1 to 4 carbon atoms and preferably denotes an ethylene group. Such polymers are described in particular in French patent 1 583 363.

(8) polymers obtained by reaction of a polyalkylene polyamine containing two primary amine groups and at least one secondary amine group with a dicarboxylic acid chosen from diglycolic acid and saturated aliphatic dicarboxylic acids having from 3 to 8 carbon atoms. The molar ratio between the polyalkylene polyamine and the dicarboxylic acid is between 0.8:1 and 1.4:1; the polyamino amide resulting therefrom is reacted with epichlorohydrin in a molar ratio of epichlorohydrin relative to the secondary amine group of the polyamino amide of between 0.5:1 and 1.8:1. Such polymers are described in particular in U.S. Pat. Nos. 3,227,615 and 2,961,347;

(9) cyclopolymers of alkyldiallylamine or of dialkyldiallylammonium such as the dimethyldiallylammonium chloride homopolymer and copolymers of diallyldimethylammonium chloride and of acrylamide;

(10) quaternary diammonium polymers with a number-average molecular mass generally of between 1000 and 100 000 such as those described, for example, in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020;

(11) polyquaternary ammonium polymers such as those especially described in patent application EP-A-122 324;

(12) quaternary polymers of vinylpyrrolidone and of vinylimidazole, such as, for example, the products sold under the names Luviquat® FC 905, FC 550 and FC 370 by the company BASF;

(13) polyamines such as Polyquart® H sold by Henkel under the reference name “Polyethylene glycol (15) Tallow polyamine” in the CTFA dictionary;

(14) Crosslinked (meth)acryloyloxy(C₁-C₄)alkyltri-(C₁-C₄)alkylammonium salt polymers such as those sold under the name Salcare® SC 92, Salcare® SC 95 and Salcare® SC 96 by the company Allied Colloids; and mixtures thereof.

Other cationic polymers that may be used in the context of the invention are cationic proteins or cationic protein hydrolysates, polyalkyleneimines, in particular polyethyleneimines, polymers containing vinylpyridine or vinylpyridinium units, condensates of polyamines and of epichlorohydrin, quaternary polyureylenes and chitin derivatives.

Among the cationic polymers mentioned above, that are suitable in the invention, the ones preferably used are quaternary cellulose ether derivatives, cationic cyclopolymers, quaternary polymers of vinylpyrrolidone and of vinylimidazole, crosslinked polymers of methacryloyloxy(C₁-C₄)alkyltri (C₁-C₄)alkylammonium salts, and cationic guar gums, and mixtures thereof.

The composition according to the invention may also comprise one or more fatty acid esters such as, for example, compounds of formula RₐCOOR_{b} in which Rₐ represents a higher fatty acid residue containing from 4 to 29 carbon atoms and R_{b} represents a hydrocarbon-based chain containing from 3 to 30 carbon atoms, such as purcellin oil (stearyl octanoate), isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate or lactate, and isostearyl neopentanoate, and mixtures thereof.

The composition according to the invention may also comprise one or more plant oils such as sweet almond oil, avocado oil, castor oil, olive oil, jojoba oil, sunflower oil, wheat germ oil, sesame oil, groundnut oil, grape seed oil, soybean oil, rapeseed oil, passion-flower oil, coconut oil, corn oil, hazelnut oil, karite butter, palm oil, apricot kernel oil and beauty-leaf oil, and mixtures thereof.

Mineral oils that may especially be mentioned include liquid paraffin and liquid petroleum jelly.

The conditioners, chosen from silicones, cationic polymers, esters, plant oils, mineral oils and synthetic oils and mixtures thereof, are preferably contained in the composition according to the invention in an amount ranging from 0.01% to 20% by weight, better still ranging from 0.1% to 10% by weight and more particularly ranging from 0.5% to 5% by weight, relative to the total weight of the composition.

The cosmetically acceptable aqueous medium may consist of water or of a mixture of water and a cosmetically acceptable solvent such as a C₁-C₄ lower alcohol, for example ethanol, isopropanol, tert-butanol or n-butanol; alkylene glycols, for instance propylene glycol; polyol ethers; C₅-C₁₀ alkanes; acetone or methyl ethyl ketone; C₁-C₄ alkyl acetates, for instance methyl acetate, ethyl acetate or butyl acetate; dimethoxyethane or diethoxyethane; and mixtures thereof.

The pH of the compositions of the invention is between 3 and 8 and preferably between 4 and 7.

The compositions according to the invention may also contain standard additives that are well known in the art, such as anionic, nonionic or amphoteric polymers, natural or synthetic anionic, amphoteric, zwitterionic, nonionic or cationic polymeric thickeners, that may or may not be associative, non-polymer thickeners, for instance acids or electrolytes, opacifiers, fragrances, colorants, organic or mineral particles, preserving agents and pH stabilizers.

A person skilled in the art will take care to select the optional additives and the amount thereof such that they do not harm the properties of the compositions of the present invention.

These additives are present in the composition according to the invention in an amount ranging from 0 to 20% by weight relative to the total weight of the composition.

The cosmetic compositions according to the invention may be in the form of fluid or thickened liquids, gels, creams, foams, simple emulsions or multiple emulsions.

They may be used, for example, as after-shampoo conditioners, rinse-off care products, deep-down care masks, or lotions or creams for treating the scalp.

According to one preferred embodiment of the invention, the composition may be used as an after-shampoo conditioner, in particular on fine hair.

The present invention also relates to a cosmetic process for treating the hair, which consists in applying an effective amount of a hair conditioning composition as described above to hair, especially fine hair, and in optionally rinsing it out after optionally leaving it on the hair for a period of time.

The examples that follow illustrate the present invention, but should not in any way be considered as limiting the invention.

### EXAMPLES

Two after-shampoo conditioning compositions are prepared from the ingredients given in the table below. Composition 1 is a composition according to the invention, while composition A is prepared for comparative purposes.

| Composition | 1 | A |
|---|---|---|
| Behentrimonium chloride as an 80% solution | 1.8% | 1.8% |
| in isopropyl alcohol/water (85/15) - | | |
| Genamin KDMP from Clariant | | |
| PEG-180 | 2.0% | 2.0% |
| Mixture of Nafol ® 1822C alcohols sold by | 3.0% | — |
| the company Condea | | |
| Cetyl alcohol | — | 3.0% |
| Cetyl esters | 0.25% | 0.25% |
| Amodimethicone, Trideceth-12, cetrimonium | 2.7% | 2.7% |
| chloride (DC 939 from Dow Corning) | | |
| Lanolin | 0.15% | 0.15% |
| Laurylmethicone-copolyol (DC 5200 from | 0.15% | 0.15% |
| Dow) | | |
| Plant extract sold under the trade name | 0.1% | 0.1% |
| MFA Complex by Barnet | | |
| Hydroxyethylcellulose | 0.2% | 0.2% |
| Pyrus malus | 0.5% | 0.5% |
| Vitamin B | 0.2% | 0.2% |
| Citric acid | 0.02% | 0.02% |
| Fragrance | qs | qs |
| Preserving agents | qs | qs |
| Water qs | 100% | 100% |

The percentages indicated in the above table are percentages by weight relative to the total weight of the composition.

The two compositions were each applied to 10 models for 1 to 5 minutes. The hair was then rinsed and dried.

The wet hair is then smoother and more supple, and the dried hair is more manageable and more individualized with the composition according to the invention.

Moreover, an analysis of the deposit of fatty alcohols onto locks of natural hair treated with 10 successive applications of the shampoo/after-shampoo conditioner combination (composition 1) relative to the shampoo/after-shampoo conditioner combination (composition A) was performed.

The fatty alcohols were then extracted from the hair with a Dionex ASE 200 extractor using dichloromethane as solvent, under the following conditions:

two 10-minute desorption cycles at 70° C.,

flush (technical term denoting the amount of solvent to be discharged, expressed as a percentage of the volume of the cell) of 60%,

pressure of 70 bar.

The amount of fatty alcohols is measured by gas chromatography, and the following results are obtained:

| | Amount of fatty alcohols (mg/g of lock) |
|---|---|
| Composition 1 | 1.5 |
| Composition A | 2.3 |

The composition according to the invention clearly gives a less substantial deposit of fatty alcohols on the hair. The hair looks markedly less charged with composition 1 when compared to hair treated with composition A.

1. Hair conditioning composition, characterized in that it comprises, in a cosmetically acceptable aqueous medium, at least one cationic surfactant, a fatty alcohol mixture that essentially comprises C₂₀₋₂₄ fatty alcohols, and optionally detergent surfactants in an amount of less than or equal to 4% by weight relative to the total weight of the composition.

2. Composition according to claim 1, characterized in that the fatty alcohol mixture comprises at least 60% by weight, preferably at least 80% by weight and more particularly at least 90% by weight, of C₂₀₋₂₄ fatty alcohols relative to the total weight of the mixture.

3. Composition according to claim 1 or 2, characterized in that the C₂₀₋₂₄ fatty alcohols are chosen from behenyl alcohol, arachidyl alcohol and lignoceryl alcohol, and mixtures thereof.

4. Composition according to any one of the preceding claims, characterized in that the fatty alcohol mixture comprises an amount of less than or equal to 40% by weight, preferably less than or equal to 20% by weight and more particularly less than or equal to 10% by weight, relative to the total weight of the mixture, of at least one C₁₄₋₁₉ alcohol.

5. Composition according to claim 4, characterized in that the C₁₄₋₁₉ alcohols are chosen from stearyl alcohol and cetyl alcohol, and mixtures thereof.

6. Composition according to any one of the preceding claims, characterized in that the fatty alcohol mixture comprises an amount of less than or equal to 40% by weight, preferably less than or equal to 20% by weight and more particularly less than or equal to 10% by weight, relative to the total weight of the mixture, of at least one C₂₅₋₄₀ alcohol.

7. Composition according to any one of the preceding claims, characterized in that the fatty alcohol mixture is contained in an amount of from 0.1% to 20% by weight and preferably from 0.5% to 10% by weight relative to the total weight of the composition.

8. Composition according to any one of the preceding claims, characterized in that the cationic surfactants are chosen from salts of optionally polyoxyalkylenated primary, secondary or tertiary fatty amines and quaternary ammonium salts, and mixtures thereof.

9. Composition according to claim 8, characterized in that the quaternary ammonium salts are chosen from:
those of general formula (I) below: 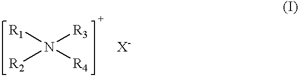
in which the symbols R₁ to R₄, which may be identical or different, represent a linear or branched aliphatic radical containing from 1 to 30 carbon atoms, or an aromatic radical such as aryl or alkylaryl; X⁻ is an anion chosen from the group of halides, phosphates, acetates, lactates, (C₂-C₆)alkyl sulphates and alkyl or alkylaryl sulphonates;
quaternary ammonium salts of imidazoline;
diquaternary ammonium salts of formula (III): 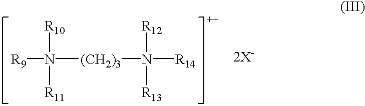
in which R₉ denotes an aliphatic radical containing from about 16 to 30 carbon atoms, R₁₀, R₁₁, R₁₂, R₁₃ and R₁₄, which may be identical or different, are chosen from hydrogen or an alkyl radical containing from 1 to 4 carbon atoms, and X⁻ is an anion chosen from the group of halides, acetates, phosphates, nitrates and methyl sulphates;
quaternary ammonium salts containing at least one ester function.

10. Composition according to any one of the preceding claims, characterized in that the cationic surfactants are chosen from behenyltrimethylammonium chloride, cetyltrimethylammonium chloride and palmitylamidopropyltrimethylammonium chloride.

11. Composition according to any one of the preceding claims, characterized in that the cationic surfactants are contained in an amount of from 0.05% to 10% by weight and preferably from 0.1% to 5% by weight relative to the total weight of the composition.

12. Composition according to any one of the preceding claims, characterized in that the detergent surfactants are chosen from anionic, nonionic and amphoteric surfactants, and mixtures thereof.

13. Composition according to any one of the preceding claims, characterized in that it also comprises at least one conditioning agent.

14. Composition according to claim 13, characterized in that the conditioning agent is chosen from silicones, cationic polymers, esters, plant oils, mineral oils and synthetic oils, and mixtures thereof.

15. Composition according to any one of the preceding claims, characterized in that the cosmetically acceptable aqueous medium consists of water or of a mixture of water and a cosmetically acceptable solvent.

16. Composition according to claim 15, characterized in that the cosmetically acceptable solvent is chosen from C₁-C₄ lower alcohols, alkylene glycols, polyol ethers, C₅-C₁₀ alkanes, acetone, methyl ethyl ketone, C₁-C₄ alkyl acetates, dimethoxyethane and diethoxyethane, and mixtures thereof.

17. Composition according to any one of the preceding claims, characterized in that it also comprises additives such as anionic, nonionic or amphoteric polymers, natural or synthetic anionic, amphoteric, zwitterionic, nonionic or cationic polymeric thickeners, that may or may not be associative, non-polymer thickeners, for instance acids or electrolytes, opacifiers, fragrances, colorants, organic or mineral particles, preserving agents and pH stabilizers.

18. Composition according to any one of the preceding claims, characterized in that it is in the form of an after-shampoo conditioner for fine hair.

19. Cosmetic process for treating the hair, characterized in that an effective amount of the hair conditioning composition according to any one of the preceding claims is applied to the hair, and the composition is optionally rinsed out after optionally leaving it on the hair for a period of time.

20. Cosmetic process for treating the hair according to claim 19, characterized in that the composition is applied to fine hair.
